(19) United States
(12) Patent Application Publication
(10) Pub. No.: US 2024/0182883 A1
(43) Pub. Date: Jun. 6, 2024

(54) INTERPENETRATING NETWORK MICROBIAL HYDROGEL WITH NATURAL POLYSACCHARIDE AND PROTEIN AND PREPARATION METHOD THEREOF

(71) Applicant: Beijing Normal University, Beijing (CN)

(72) Inventors: Anjie LI, Beijing (CN); Danyang WANG, Beijing (CN)

(21) Appl. No.: 18/225,937

(22) Filed: Jul. 25, 2023

(30) Foreign Application Priority Data
Dec. 6, 2022 (CN) 2022115611214

(51) Int. Cl. C12N 11/04; B33Y 70/00; C12N 1/12; C12N 1/20

(52) U.S. Cl. CPC C12N 11/04; B33Y 70/00; C12N 1/12; C12N 1/20

(57) ABSTRACT

The present disclosure belongs to the technical field of biomaterials and microbial immobilization, and relates to an interpenetrating network (IPN) microbial hydrogel with natural polysaccharides (PS) and proteins (PN) and a preparation method thereof. The natural PS of the hydrogel is selected from alginates, and the natural PN is selected from fibrous protein silk fibroin (SF) or methacrylated SF (SilMA). The natural PS and PN mimic extracellular PS and PN of aerobic granular sludge (AGS). Components in the hydrogel are uniformly mixed and each crosslinked to form an IPN. The natural PS undergoes ionic crosslinking, the SF in the natural PN undergoes self-assembly to form physical crosslinking, or the SilMA undergoes photocrosslinking. Loaded microbes uniformly adhere to a structure of the hydrogel. According to the preparation method of the hydrogel, a hydrogel precursor solution is sonicated and mixed with microbes, followed by bioprinting combined with crosslinking.

### CROSS REFERENCE TO RELATED APPLICATION

This patent application claims the benefit and priority of Chinese Patent Application No. 2022115611214 filed with the China National Intellectual Property Administration on Dec. 6, 2022, the disclosure of which is incorporated by reference herein in its entirety as part of the present application.

### TECHNICAL FIELD

The present disclosure belongs to the technical field of biomaterials and microbial immobilization, and relates to an interpenetrating network (IPN) microbial hydrogel with natural polysaccharides (PS) and proteins (PN) and a preparation method thereof.

### BACKGROUND

Microbial remediation technologies can partly or completely convert environmental organic pollutants into stable and nontoxic final products, featuring safety, high efficiency, low energy consumption and environmental friendliness. However, lacking long-term operational stability of the system and difficultly in microbes collection and recycle become the principal constraints in current technical engineering applications. An idea of artificially combining functional microbes with biomanufacturing to mimic a natural system is emerging. Conventional microbial immobilization technologies have been successfully used in the field of environmental remediation, which can realize high biomass and high microbial survival rate, facilitate biomass recycling, and targeted select functional microbes to rapidly assemble and thus remedy different target pollutants. In spite of broad prospects, there are still challenges. In one aspect, interactions between encapsulation materials and encapsulated microbes are neglected, which are critical to the prediction, optimization and improvement of performance and long-term usability of the system. In another aspect, traditional manufacturing poses a limitation to the shape control of the product, thereby influencing the application range and treatment effect of the process.

Bioprinting technology, using living cells mixed with biomaterials to prepare bioink, is enable the rapid on-demand production of bioactive structures with specific functions, and opens a new path for artificial construction of a microbial system. Hydrogel as a commonly preferred ink material for bioprinting has high porosity and permeability, providing a favorable environment for cell growth. Hydrogels can be classified into the following two categories based on their polymer source: natural polymer-based hydrogels are formed by crosslinking of natural polymer materials such as PS and PN, with excellent biocompatibility and biodegradability; and synthetic polymer-based hydrogels are prepared from synthetic polymers such as polyvinyl alcohol (PVA) and polyethylene glycol (PEG), with a mechanical strength advantage.

Traditional single-component crosslinked hydrogels usually cannot satisfy a plurality of requirements for bioprinting. The stability, mechanical properties, and biocompatibility of IPN hydrogels with dual- or multi-component polymers are substantially enhanced. In the field of medical engineering, China Patent CN202111038821.0 discloses a high-comprehensive-performance photocuring biological 3D printing composite hydrogel and a preparation method and use thereof. The gel formed by covalent crosslinking of methylacryloyl gelatin, hyaluronic acid and silk fibroin (SF) with high biocompatibility, high mechanical strength and rapid gelation can be used for preparing spinal cord stents, which can inoculate neuron-carrying cells or carry out neuron-carrying cell printing. In the field of hyaluronic acid preparation, China Patent CN202210130465.3 discloses a method for immobilizing microbial to yield hyaluronic acid in high level using 3D printing technology. Streptococcus equi subsp. for producing hyaluronic acid is loaded into a bioink based on GelMA, and a gel mesh is prepared through 3D printing technology, then cultured in a fermentation broth. Compared with planktonic microorganisms, this method increases the yield of hyaluronic acid, and bacteria are easy to isolate and recover. In the field of bioelectrochemistry, China Patent CN202210169886.7 discloses a 3D printing bioink, a preparation method thereof, a 3D printing biocathode material, and a preparation method and use thereof. A conductive bioink is prepared by mixing sodium alginate (SA), cellulose, acetylene black, Shewanella oneidensis, and liquid medium. The conductive bioink is 3D printed and crosslinked to prepare a biocathode material with high bacterial concentration and excellent extracellular electron transfer capability, which has very excellent degradation capability on toxic organic matters in sewage.

Microbial printing study is very limited and the technology gaps of which remain. There is still an urgent need for developing a microbially loaded bioink that accommodates environmental remediation to print an eco-friendly multi-network polymer hydrogel that has high mechanical integrity and stability, is suitable for various polluted environments, and stimulates the cell adhesion ability. The development and application of advanced technologies is inspired by self-assembly of microbial communities into a complex and stable ecosystem and the creature survival mode in nature. The aerobic granular sludge (AGS) of the novel biological wastewater treatment process forms a spherical three-dimensional structure with a compact and regular shape by effects of multi-microorganisms. Moreover, it has been demonstrated by rheological characterization that the AGS is substantially a microbial hydrogel bead formed by crosslinking of extracellular PS and PN. It is still difficult to prepare a hydrogel material that accommodates microbial remediation and has excellent comprehensive properties by using “natural assembly” to guide “artificial synthesis” from the bottom up.

### SUMMARY

To solve the problems existed in the prior art, in the present disclosure, by mimicking naturally self-assembled extracellular polymer substance of AGS with mixed natural PS and PN, an IPN microbial hydrogel with natural PS and PN that possesses excellent structural stability and bioactivity is developed based on bioprinting. The IPN microbial hydrogel provided by the present disclosure can be used in the field of microbial remediation. In one aspect, rapid, stable and shape-controllable microbial encapsulation and immobilization can be realized. In another aspect, artificial bioprinting mimicking an ecosystem can be used in basic research on micro-level microbial interactions, realizing adjustable and controllable structure and function of microbial communities.

To achieve the above objectives, the present disclosure is achieved by the following product and technical solutions:

An IPN microbial hydrogel with natural PS and PN is provided. The hydrogel is prepared from a mixture of natural PS and PN hybrid microbial suspension through a bioprinting device; in the hydrogel, the natural PS and PN are uniformly mixed and each crosslinked to form an IPN; the microbial suspension is selected from bacteria or microalgae with a cell concentration of 10⁶-10⁹ cells/mL; and loaded microbes uniformly interpenetrate and adhere to a structure of the hydrogel.

According to the IPN microbial hydrogel with natural PS and PN, where the natural PS and PN mimic extracellular PS and PN of AGS, the natural PS is selected from the group consisting of alginates, and the natural PN is selected from the group consisting of fibrous protein SF and methacrylated SF (SilMA); and the natural PS and PN has a mass ratio of 1:(5-30).

The alginates as natural water-soluble linear polysaccharides mimic polysaccharide components in the AGS. The alginates can chelate with metal ions (Ca, Ba, etc.) to form a hydrogel rapidly. In the field of environment, the alginates have been widely used for encapsulation of microbes, enzymes and other substances to remove a plurality of pollutants including dyes, heavy metals and antibiotics. However, crosslinking between the alginates and the metal ions depends on noncovalent force. Existing ion exchange process will lead to hydrogel swelling and disintegration, so the service life of the alginates is an urgent problem to be solved.

The SF as one of the fibrous proteins widely present in nature mimics the PN component of the AGS. Most of the fibrous proteins are structural proteins, which form a fibrous or clubbed secondary structure rich in single type by connecting long amino acid peptide chains, having functions of maintaining cell morphology, mechanical support, and weight bearing. Herein, the SF has highly repeated amino acid sequences, and these repeat units can form a densely arranged and highly structurally ordered j-sheet crystalline region under environmental stimuli of temperature, pH, solvent and stress, giving unique mechanical and structural support performance. In addition, the SF has excellent biocompatibility and biodegradability. SilMA prepared from glycidyl methacrylate modified SF can regulate mechanical properties based on the degree of modification, and possesses photocrosslinking properties to enable rapid gelation.

According to the IPN microbial hydrogel with natural PS and PN, where the IPN is formed from the natural PS and PN that uniformly interpenetrate and each crosslink, the natural PS undergoes ionic crosslinking, and the SF in the natural PN undergoes self-assembly to form physical crosslinking or the SilMA undergoes photocrosslinking.

According to the IPN microbial hydrogel with natural PS and PN, where the IPN microbial hydrogel with natural PS and PN is suitable for rapid and shape-controllable microbial encapsulation and immobilization in the field of microbial remediation and basic research on microbial interactions in a synthetic microbial system, with structural stability and bioactivity.

The present disclosure provides a preparation method of an IPN microbial hydrogel with natural PS and PN, including the following steps:

step 1, dissolving alginate and SF (or SilMA) in a solvent to prepare a hydrogel precursor solution;

step 2, subjecting the hydrogel precursor solution to sonication;

step 3, successively adding a microbial suspension and a photoinitiator (optional) in a sonicated hydrogel precursor solution, and gently and uniformly mixing to prepare a bioink;

step 4, filling the bioink into a bioprinting device to obtain a bioprinted structure;

step 5, crosslinking the bioprinted structure to obtain an IPN microbial hydrogel.

Preferably, in the step 1, the hydrogel precursor solution consists of alginate in a final concentration by weight of 1-1.5% w/v mixed with SF (or SilMA) in a final concentration by weight of 10-30% w/v.

Preferably, in the step 1, the solvent is selected from the group consisting of pure water and a microbial culture medium.

Preferably, in the step 2, the sonication is conducted at an amplitude of 20-70% for 30-90 s; and the hydrogel precursor solution is sonicated again under the same conditions after 15-30 min of standing.

Preferably, in the step 3, the microbial suspension is collected from a cell suspension cultured to a stationary phase, centrifuged at 6,000-8,000 rpm for 5-10 min to remove a culture medium, and re-suspended in the solvent.

Preferably, in the step 3, the photoinitiator is lithium phenyl-2,4,6-trimethylbenzoylphosphinate (LAP); 0.1-0.2% photoinitiator is added only if the SilMA is used; and the bioink is kept in the dark after addition.

Preferably, in the step 4, the bioprinting device is selected from the group consisting of a commercial three-dimensional (3D) printer and a simple self-assembled bioprinting device.

Preferably, in the step 5, for the crosslinking, an alginate-SF-containing bioink undergoes ionic crosslinking, and an alginate-SilMA-containing bioink undergoes the ionic crosslinking and an subsequent photocrosslinking; the ionic crosslinking refers to the immersion of the bioprinted structure in 4% w/v CaCl₂) or BaCl₂ for crosslinking reaction for 2-4 h; and the photocrosslinking refers to the exposure of the bioprinted structure to 365-405 nm ultraviolet (UV) light at a power density of 10-50 mW/cm² for 30-180 s.

Compared with the prior art, the present disclosure has the following advantages and beneficial effects:

1. The present disclosure selects natural polymer materials from nature, mimics extracellular polymer substance of naturally self-assembled AGS by mixing natural PS and fibrous protein, and provides an excellent growth and living environment for artificially biomanufactured synthetic microbial communities.

2. Natural PS in the present disclosure is selected from alginate, which is widely sourced and low-cost, and can also be extracted from residual sludge to facilitate the recycling economy.

3. According to the IPN hydrogel with alginate and SF provided by the present disclosure, an IPN hydrogel completely constructed by physical crosslinking is prepared by alginate ionic crosslinking plus SF self-assembly. The process is simple, and crosslinking methods are green and eco-friendly, overcoming the toxicity brought by chemical crosslinking agents while solving the problem of poor effects of physical crosslinking agents through a dual network.

4. With photocrosslinking properties, high printing precision, and excellent formability, an IPN hydrogel with alginate and SilMA provided by the present disclosure can construct a plurality of hydrogels with complex structure by bioprinting technology. The hydrogels can be used in wide application fields that hydrogels of any size and shape can be prepared for basic research on microbial encapsulation and immobilization in the field of microbial remediation and microbial interactions in a synthetic microbial system.

### BRIEF DESCRIPTION OF THE DRAWINGS

FIG. 1 shows a flow chart of preparation of an IPN microbial hydrogel with natural PS and PN provided by the present disclosure.

FIG. 2 shows schematic illustrations of printing devices used in Example 1 and Comparative Example 2 of the present disclosure.

FIGS. 3A-G show scanning electron micrographs (SEM) (a to c) and cross-sectional structural diagram (d to g) of the hydrogels provided by the present disclosure.

FIG. 4 shows a chart of the swelling ratio of hydrogels in synthetic wastewater versus time provided in Examples 1 to 2 and Comparative Examples 1 to 3.

FIGS. 5A-B show FTIR spectra (a) of hydrogels in an initial run and after 7 days of operation in synthetic wastewater and a histogram (b) of R-sheet content after 7 days of operation provided in Examples 1 to 2 and Comparative Example 3.

FIGS. 6A-C show SEM images of bacterial distribution in hydrogels provided in Example 1 and Comparative Examples 1 to 2.

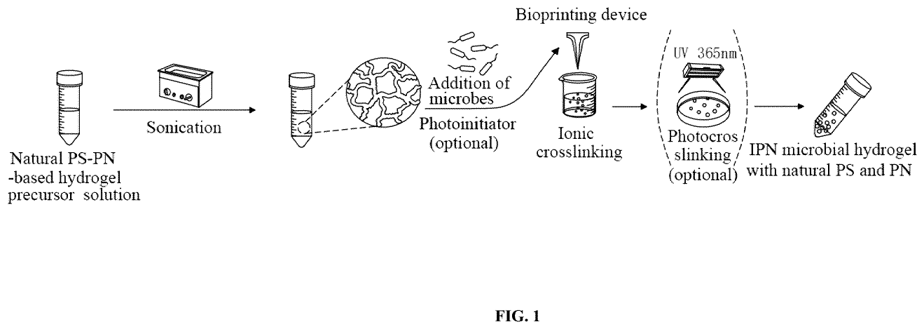

### DETAILED DESCRIPTION OF THE EMBODIMENTS

The above content of the present disclosure will be further described in detail below in conjunction with specific examples, but they should not be construed that the scope of the above subject of the present disclosure is only limited to the following examples. Any of the same method realized based on the above subject of the present disclosure falls within the scope of the present disclosure.

### Example 1

Preparation of a Bacteria-Loaded Photocrosslinked IPN Microbial Hydrogel with Natural PS and PN

Step 1: Preparation of SilMA: 4% (w/v) silkworm cocoon pieces were placed and boiled in 0.05 M Na₂CO₃ solution at 100° C. for 30 min to remove the silk gum, followed by rinsing with distilled water to obtain a product. The degummed product was squeezed dry and placed in an oven overnight to dry to obtain a dried matter. The following day, 20% (w/v) of the dried matter was dissolved in 9.3 M lithium bromide (LiBr) solution at 60° C. for 1 h. After full dissolution, 424 mM glycidyl methacrylate (GMA) was gradually dropped in and stirred at 60° C. for 6 h at 300 rpm. Next, the resulting solution was filtered and dialyzed against distilled water for 5-7 days using a 12-14 kDa dialysis membrane, and the water was changed 3 times daily. Finally, the solution was filtered to remove insolubles and lyophilized to obtain SilMA, which was stored at 4° C. for later use.

Step 2: Preparation of a hydrogel precursor solution: 1.5% (w/v) SA and 20% (w/v) SilMA were dissolved in distilled water to prepare a hydrogel precursor solution, which was subjected to repeated heat treatments (3 times for 30 min each) at 70° C. and cooling down to room temperature to achieve a simple sterilization.

Step 3: Sonication of the hydrogel precursor solution: The hydrogel precursor solution was sonicated in an ultrasonic cleaner at 50% amplitude for 30 s. After 15 min of standing, the hydrogel precursor solution was sonicated again under the same condition.

Step 4: Preparation of a cell suspension: Pseudomonas aeruginosa PAO1 for bioprinting was incubated in LB medium at an inoculum size of 1% and cultured at 180 rpm for 24 h at 37° C. till the stationary phase. The culture medium was removed by centrifugation at 6,000 rpm for 7 mn, and the obtained cell pellets were resuspended in distilled water.

Step 5: Preparation of a bioink capable of photocrosslinking: The cell suspension and 0.2% (w/v) photoinitiator LAP were added successively in the sonicated hydrogel precursor solution and gently and uniformly mixed in the dark.

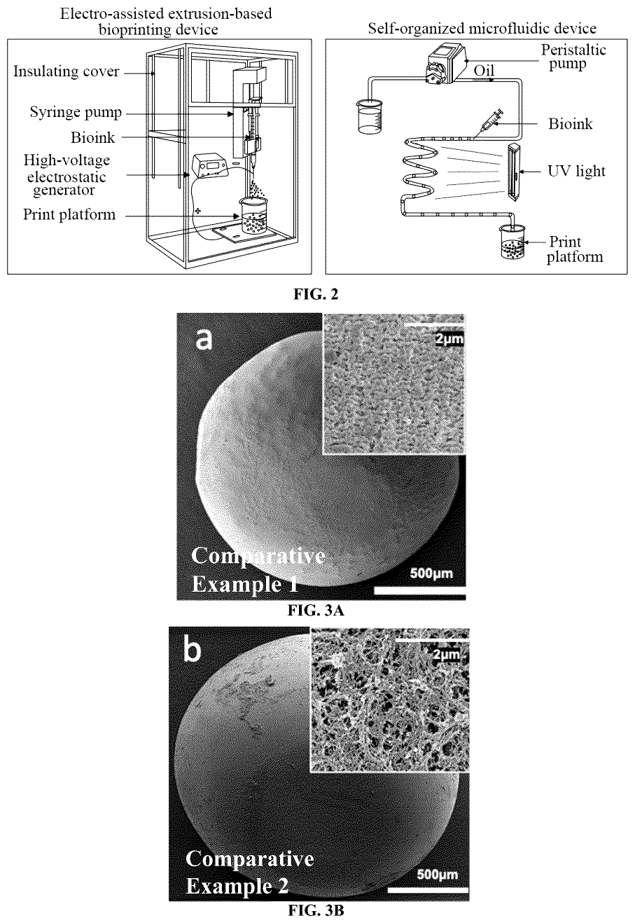

Step 6: Bioprinting of an IPN microbial hydrogel capable of photocrosslinking (FIG. 2): A simply laboratory-assembled electro-assisted extrusion-based bioprinting device was used.

The bioink was filled into a 10 mL syringe and pumped through a microsyringe at a flow rate of 50 mL/h to a printer nozzle (25G). The diameter of printed hydrogel beads was controlled by regulating the electrostatic field generated by the high-voltage electrostatic generator (8-12 kV) according to the viscosity of the bioink.

Step 7: Crosslinking treatment of the IPN microbial hydrogel capable of photocrosslinking: The printed hydrogel beads were directly immersed in 4% (w/v) CaCl₂) for the ionic crosslinking reaction for 2 h. Subsequently, the printed hydrogel beads were taken out and exposed to 365 nm UV light at a power density of 20 mW/cm² for 180 s.

### Example 2

Preparation of a Bacteria-Loaded IPN Microbial Hydrogel with Natural PS and PN

Step 1: Preparation of SF: 4% (w/v) silkworm cocoon pieces were placed and boiled in 0.05 M Na₂CO₃ solution at 100° C. for 30 min to remove the silk gum, followed by rinsing with distilled water to obtain a product. The degummed product was squeezed dry and placed in an oven overnight to dry to obtain a dried matter. The following day, 20% (w/v) of the dried matter was dissolved in 9.3 M lithium bromide (LiBr) solution at 60° C. for 1 h. Next, the resulting solution was filtered and dialyzed against distilled water for 5-7 days using a 12-14 kDa dialysis membrane, and the water was changed 3 times daily. Finally, the solution was filtered to remove insolubles and lyophilized to obtain SF, which was stored at 4° C. for later use.

Step 2: Preparation of a hydrogel precursor solution: 1.5% (w/v) SA and 20% (w/v) SF were dissolved in distilled water to prepare a hydrogel prepolymer solution, which was subjected to repeated heat treatments (3 times for 30 min each) at 70° C. and cooling down to room temperature to achieve a simple sterilization.

Step 3: Sonication of the hydrogel prepolymer solution: This step was consistent with step 3 in Example 1.

Step 4: Preparation of a cell suspension: This step was consistent with step 4 in Example 1.

Step 5: Preparation of a bioink: The cell suspension was added in the sonicated hydrogel precursor solution and gently and uniformly mixed.

Step 6: Bioprinting of an IPN microbial hydrogel: This step was consistent with step 6 in Example 1.

Step 7: Crosslinking treatment of the IPN microbial hydrogel: The printed hydrogel beads were directly immersed in 4% (w/v) CaCl₂) for the ionic crosslinking reaction for 2 h.

### Example 3

Preparation of a microalgae-loaded photocrosslinked IPN microbial hydrogel with natural PS and PN

Step 1: Preparation of SilMA: This step was consistent with step 1 in Example 1.

Step 2: Preparation of a hydrogel precursor solution: This step was consistent with step 2 in Example 1.

Step 3: Sonication of the hydrogel precursor solution: This step was consistent with step 3 in Example 1.

Step 4: Preparation of a cell suspension: Microalgae Chlorella sp. for bioprinting was incubated in BG11 Medium at an inoculum size of 10% and cultured in an illumination incubator at a light intensity of 2,000 Lux for around two weeks at 25° C. under a 12 h/12 h light-dark cycle till the stationary phase. The cells were shaken twice a day at regular times. The culture medium was removed by centrifugation at 6,000 rpm for 7 min, and the obtained cell pellets were resuspended in distilled water.

Step 5: Preparation of a bioink capable of photocrosslinking: This step was consistent with step 5 in Example 1.

Step 6: Bioprinting of an IPN microbial hydrogel capable of photocrosslinking: This step was consistent with step 6 in Example 1.

Step 7: Bioprinting of the IPN microbial hydrogel capable of photocrosslinking: This step was consistent with step 7 in Example 1.

### Comparative Example 1

### Preparation of Single-Component SA Hydrogel

Step 1: Preparation of a hydrogel precursor solution: 1.5% (w/v) SA was dissolved in distilled water to prepare a hydrogel precursor solution, which was subjected to repeated heat treatments (3 times for 30 min each) at 70° C. and cooling down to room temperature to achieve a simple sterilization.

Step 2: Preparation of a cell suspension: This step was consistent with step 4 in Example 1.

Step 3: Preparation of a bioink: The cell suspension was added in the hydrogel precursor solution and gently and uniformly mixed.

Step 4: Bioprinting of an SA single-network hydrogel: This step was consistent with step 6 in Example 1.

Step 5: Crosslinking treatment of the SA single-network hydrogel: The printed hydrogel beads were directly immersed in 4% (w/v) CaCl₂) for the ionic crosslinking reaction for 2 h.

### Comparative Example 2

### Preparation of Single-Component SF Hydrogel

Step 1: Preparation of SilMA: This step was consistent with step 1 in Example 1.

Step 2: Preparation of a hydrogel precursor solution: 30% (w/v) SilMA was dissolved in distilled water to prepare a hydrogel precursor solution, which was subjected to repeated heat treatments (3 times for 30 min each) at 70° C. and cooling down to room temperature to achieve a simple sterilization.

Step 3: Preparation of a cell suspension: This step was consistent with step 4 in Example 1.

Step 4: Preparation of a bioink: The cell suspension and 0.2% (w/v) photoinitiator LAP were added successively in the sonicated hydrogel precursor solution and gently and uniformly mixed in the dark.

Step 5: Bioprinting and crosslinking of an SF single-network hydrogel (FIG. 2): An SF single-network bioink was bioprinted by using a self-organized simple microfluidic device. The bioink and oil were pumped at appropriate flow rates into a syringe and silicone tube as the dispersed and continuous phases, respectively. The self-organized microfluidic device consisted of a printer nozzle (25G) inserted at a certain angle into a transparent silicone tube. The external oil exerts a shear stress on the internal bioink to form microdroplets at the exit of the nozzle, which were subsequently fully photocrosslinked as they traveled through a sufficiently long distance in the silicone tube. It was conducted under 365 nm UV light at a power density of 20 mW/cm² for 180 s.

### Comparative Example 3

### Preparation of Dual-Component Hydrogel Capable of Photocrosslinking

Step 1: Preparation of SilMA: This step was consistent with step 1 in Example 1.

Step 2: Preparation of a hydrogel precursor solution: This step was consistent with step 2 in Example 1.

Step 3: Preparation of a cell suspension: This step was consistent with step 4 in Example 1.

Step 4: Preparation of a bioink capable of photocrosslinking: This step was consistent with step 5 in Example 1.

Step 5: Bioprinting of a dual-component hydrogel capable of photocrosslinking: This step was consistent with step 6 in Example 1.

Step 6: Crosslinking treatment of the dual-component hydrogel capable of photocrosslinking: This step was consistent with step 7 in Example 1.

Advantages of IPN microbial hydrogels in examples over those in comparative examples in microbial remediation will be illustrated below through several tests.

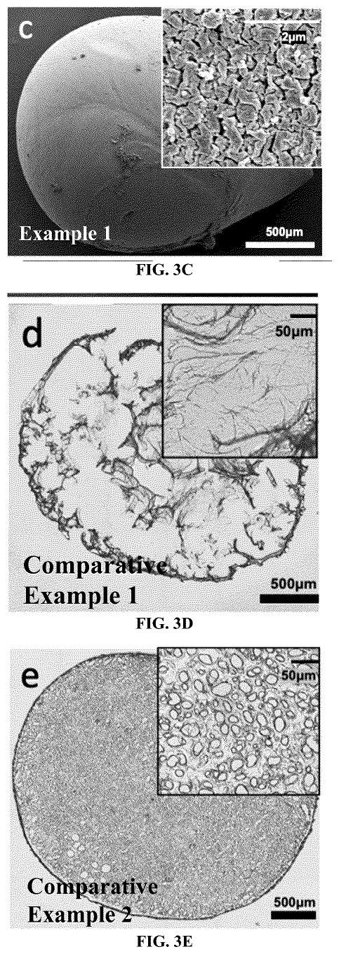

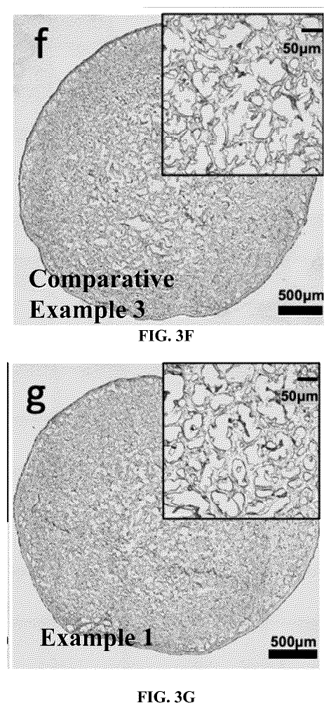

1. Microscopic morphology of hydrogel (FIGS. 3A-G): SEM shows that the overall microstructure of newly prepared hydrogel beads appears as various degrees of pitted surface similar to the lunar surface. The single-component SA hydrogel in Comparative Example 1 has a compact matrix and small pores. The single-component SF hydrogel in Comparative Example 2 has a uniform and interconnected porous honeycomb microstructure. The IPN hydrogel in Example 1 appears as a wrinkle surface and pores between two single-component gels. The cross-sectional slices of the hydrogels are further observed. The hydrogel in Comparative Example 1 appears a loss of slice structure due to the weak structure. For the hydrogel in Comparative Example 2, inner pores are larger than surface ones, approximately 10-50 μm in diameter. The sectional view of the hydrogel in Comparative Example 3 reveals an uneven internal structure, a loose inner layer, a compact outer layer, and weak connectivity of the porous region. By comparing the hydrogel in Example 1 with that in Comparative Example 3, the structural uniformity and the connectivity are significantly enhanced.

The hydrogels in Examples 1 to 2 and Comparative Examples 1 to 3 were run in the synthetic wastewater, and the long-term structural stability and biocompatibility of hydrogel materials were investigated. With 50 mL conical flasks as reactors, hydrogel beads were incubated with 15% inoculum at 85 rpm and 25° C.

The synthetic wastewater simulating municipal domestic sewage was used, and its composition was as follows: COD 200 mg/L (C₆H₁₂O₆); 40 mg NH₄⁺-N/L (NH₄Cl); 5 mg PO₄³⁻-P/L (KH₂PO₄); 300 mg Na⁺/L (NaHCO₃); 10 mg Ca²⁺/L (CaCl₂)); 5 mg Mg²⁺/L (MgSO₄·7H₂O); 1 ml/L trace elements consisted of: 1.5 g/L FeCl₃·6H₂O, 0.15 g/L H₃BO₃, 0.03 g/L CuSO₄·5H₂O, 0.18 g/L KI, 0.12 g/L MnCl₂·H₂O, 0.06 g/L Na₂MoO₄·2H₂O, 0.12 g/L ZnSO₄·7H₂O, 0.15 g/L CoCl₂·6H₂O, and 10 g/L EDTA. The pH was adjusted to 7.0-7.5 by using 1 M HCl.

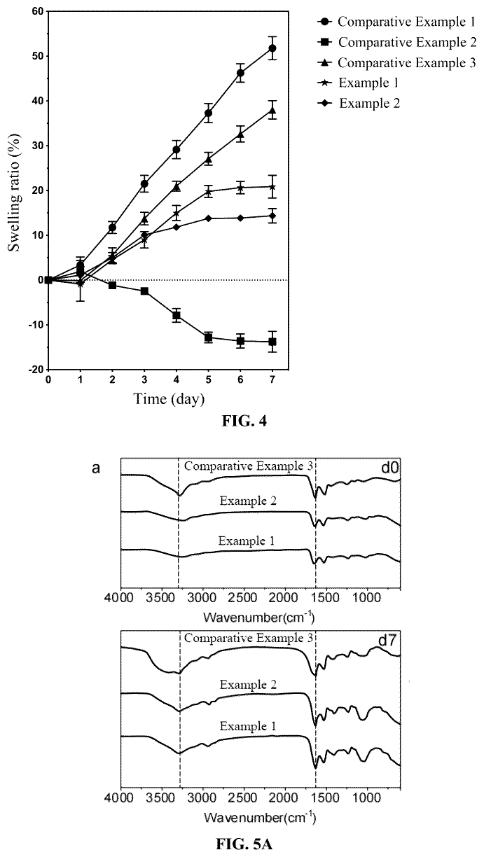

2. Swelling ratio of hydrogels (FIG. 4): The swelling ratio of single-component SA hydrogel in Comparative Example 1 is far higher than that of the remaining groups and has been on the rise, which tops out at nearly 60% until broken. The single-component SF hydrogel in Comparative Example 2 shows a negative swelling ratio, indicating its excessive self-assembly leads to the hardening shrinkage of the hydrogel. The mixing of SF with SA leads to a decrease in swelling. However, the dual-component hydrogel capable of photocrosslinking in Comparative Example 3 still shows a continuous rise in swelling. The swelling of sonicated IPN hydrogels in Examples 1 and 2 substantially remains constant after Day 5, which is far lower than that in the unsonicated group (<20%).

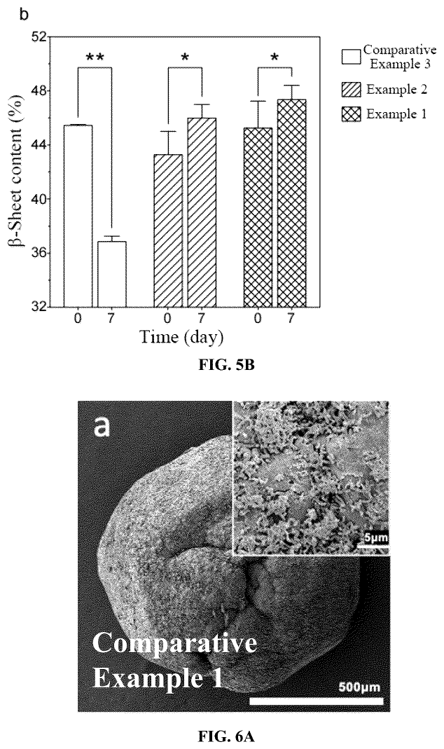

3. Structural changes of hydrogel molecule: The molecular conformation changes of SF in Examples 1, 2 and Comparative Example 3 are described by Fourier transform infrared spectroscopy (FTIR) and Fourier self-deconvolution (FSD) curve fitting (FIGS. 5A-B). Hydrogels from these three groups show wider peak values at around 3281 cm⁻¹, particularly IPN hydrogels in Examples 1 and 2 after sonication pretreatment, indicating intensive hydrogen-bonding interactions between SF and SA. Both the IPN hydrogels in Examples 1 and 2 after sonication pretreatment show enhanced amide peak I (1625 cm⁻¹), and the R-sheet content significantly increases by 6.24% and 4.63% after 7 days of running, respectively. The R-sheet content of the unsonicated dual-component hydrogel in Comparative Example 1 significantly decreases within 7 days. This indicates that sonication pretreatment not only increases the intermolecular chain entanglement between SF and SA, but also induce the subsequent self-assembly of SF molecules to promote the maintenance of the mechanical strength of the hydrogel.

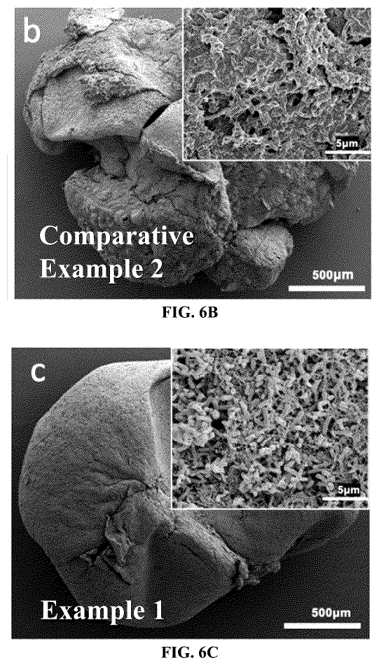

4. Microbial proliferation in hydrogels (FIGS. 6A-C): After 7 days of running, representative images of bacterial adhesion to hydrogels are characterized by SEM. The compact structure of the single-component SA hydrogel in Comparative Example 1 is destroyed, and substantial masses of colonies spring from cracks on the material surface. Only a small quantity of bacteria adhere to the complete gel structure remained on the surface of hydrogel beads, and there is a large bacteria-free blank area. After 7 days of culture of bacteria in the single-component SF hydrogel in Comparative Example 2, only a few bacteria with clear contour can be observed, most of the bacteria are intertwined with the gel structure to present a boundless state, and indistinguishable matrix is a gel structure or secreted by bacteria. In the IPN hydrogel in Example 1, the bacteria are dense with clear contour and are uniformly embedded into the gel network structure with a visible linkage of bacteria to the material, indicating that the IPN hydrogel provides an appropriate growth support environment for bacteria.

What is claimed is:

1. An interpenetrating network (IPN) microbial hydrogel with natural polysaccharides (PS) and proteins (PN), wherein the hydrogel is prepared from a mixture of natural PS and PN hybrid microbial suspension through a bioprinting device; in the hydrogel, the natural PS and PN are uniformly mixed and each crosslinked to form an IPN; the microbial suspension is selected from bacteria or microalgae with a cell concentration of 10⁶-10⁹ cells/mL; and loaded microbes uniformly interpenetrate and adhere to a structure of the hydrogel.

2. The IPN microbial hydrogel with natural PS and PN according to claim 1, wherein the natural PS and PN mimic extracellular PS and PN of aerobic granular sludge (AGS), the natural PS is selected from the group consisting of alginates, and the natural PN is selected from the group consisting of fibrous protein silk fibroin (SF) and methacrylated SF (SilMA); and the natural PS and PN has a mass ratio of 1:(5-30).

3. The IPN microbial hydrogel with natural PS and PN according to claim 1, wherein the IPN is formed from the natural PS and PN that uniformly interpenetrate and each crosslink, the natural PS undergoes ionic crosslinking, and the SF in the natural PN undergoes self-assembly to form physical crosslinking or the SilMA undergoes photocrosslinking.

4. The IPN microbial hydrogel with natural PS and PN according to claim 1, wherein the IPN microbial hydrogel with natural PS and PN is suitable for rapid and shape-controllable microbial encapsulation and immobilization in a technical field of microbial remediation and basic research on microbial interactions in a synthetic microbial system, with structural stability and bioactivity.

5. A method for preparing the IPN microbial hydrogel with natural PS and PN according to claim 1, comprising the following steps:
step 1, dissolving alginate and SF (or SilMA) in a solvent to prepare a hydrogel precursor solution;
step 2, subjecting the hydrogel precursor solution to sonication;
step 3, successively adding a microbial suspension and a photoinitiator (optional) in a sonicated hydrogel precursor solution, and gently and uniformly mixing to prepare a bioink;
step 4, filling the bioink into a bioprinting device to obtain a bioprinted structure;
step 5, crosslinking the bioprinted structure to obtain an IPN microbial hydrogel.

6. The method according to claim 5, wherein in the step 1, the hydrogel precursor solution comprises alginate in a final concentration by weight of 1-1.5% w/v mixed with SF (or SilMA) in a final concentration by weight of 10-30% w/v.

7. The method according to claim 5, wherein in the step 1, the solvent is selected from the group consisting of pure water and a microbial culture medium.

8. The method according to claim 5, wherein in the step 2, the sonication is conducted at an amplitude of 20-70% for 30-90 s; and the hydrogel precursor solution is sonicated again under the same conditions after 15-30 min of standing.

9. The method according to claim 5, wherein in the step 3, the microbial suspension is collected from a cell suspension cultured to a stationary phase, centrifuged at 6,000-8,000 rpm for 5-10 min to remove a culture medium, and re-suspended in the solvent.

10. The method according to claim 5, wherein in the step 3, the photoinitiator is lithium phenyl-2,4,6-trimethylbenzoylphosphinate (LAP); 0.1-0.2% photoinitiator is added only if the SilMA is used; and the bioink is kept in the dark after addition.

11. The method according to claim 5, wherein in the step 4, the bioprinting device is selected from the group consisting of a commercial three-dimensional (3D) printer and a simple self-assembled bioprinting device.

12. The method according to claim 5, wherein in the step 5, for the crosslinking, an alginate-SF-containing bioink undergoes ionic crosslinking, and an alginate-SilMA-containing bioink undergoes ionic crosslinking and an subsequent photocrosslinking; the ionic crosslinking refers to the immersion of the bioprinted structure in 4% w/v CaCl₂) or BaCl₂ for crosslinking reaction for 2-4 h; and the photocrosslinking refers to the exposure of the bioprinted structure to 365-405 nm ultraviolet (UV) light at a power density of 10-50 mW/cm² for 30-180 s.
